(19) United States
(12) Patent Application Publication
(10) Pub. No.: US 2026/0232343 A1
(43) Pub. Date: Aug. 13, 2026

(54) CATHETERS WITH TELESCOPING CUTTING TOOL AND ASPIRATION

(71) Applicant: BARD PERIPHERAL VASCULAR, INC., Franklin Lakes, NJ (US)

(72) Inventor: Germán LOESENER, Saratoga Springs, UT (US)

(73) Assignee: BARD PERIPHERAL VASCULAR, INC., Franklin Lakes, NJ (US)

(21) Appl. No.: 19/160,761

(22) PCT Filed: Mar. 28, 2023
(86) PCT No.: PCT/US2023/016568

§ 371 Date: Aug. 28, 2025

(51) Int. Cl. A61B 17/3207; A61B 17/00

(52) U.S. Cl. CPC A61B 17/320758; A61B 2017/00991; A61B 2217/005

(57) ABSTRACT

Disclosed herein is a intravascular cutting tool for an occlusion removal procedure. The intravascular cutting tool includes a catheter body and a telescoping cutting assembly. The telescoping cutting assembly includes an outer blade coupled to and 2024/205575 extending distally from the catheter body and at least one an inner blade positioned radially inward of the outer blade. The at least one inner blade is axially moveable between a retracted configuration and a cutting configuration. The at least one inner blade extends distally beyond the outer blade when in the cutting configuration than in the retracted configuration, and the outer blade and the at least one inner blade cooperatively define a tapered cutting profile.

### TECHNICAL FIELD

The present specification generally relates to intravascular cutting tools and, more specifically, intravascular cutting tools for removal of occlusions within a vessel.

### BACKGROUND

The build-up of occlusive material (such as atheromatous plaque within the arterial wall, the formation of thrombi, a clot, etc.) in a blood vessel may lead to serious circulatory issues. For example, when a complete blockage occur, tissues distal of the blockage may be deprived of oxygen and nutrients, thereby leading to death of those cells. In other cases, an atheroma or other type of stenotic lesion in a peripheral vein or artery can have a corresponding effect on tissue and cells supplied by the blocked blood vessel. In yet another example, atheromatous plaque build-up in a coronary artery may lead to a coronary infarction, especially when the artery is so narrowed by the plaque that a clot or thrombus cannot pass there through.

Accordingly, a need exists for improved devices and methods that remove occlusions within a vessel.

### SUMMARY

In one embodiment, an intravascular cutting tool for an occlusion removal procedure is disclosed. The intravascular cutting tool may include a catheter body and a telescoping cutting assembly. The telescoping cutting assembly may include an outer blade coupled to and extending distally from the catheter body and at least one inner blade positioned radially inward of the outer blade, the at least one inner blade being axially moveable between a retracted configuration and a cutting configuration, wherein the at least one inner blade extends more distally beyond the outer blade when in the cutting configuration than in the retracted configuration, and wherein the outer blade and the at least one inner blade cooperatively define a tapered cutting profile.

In another embodiment, a method for atherectomy or thrombectomy procedure is disclosed. The method may include: advancing an intravascular cutting tool to an intervention site of a blood vessel, the intravascular cutting tool including: a catheter body, and a telescoping cutting assembly comprising an outer blade coupled to and extending distally from the catheter body and at least one inner blade positioned radially inward of the outer blade, the at least one inner blade being axially moveable between a retracted configuration and a cutting configuration, wherein the at least one inner blade extends distally beyond the outer blade when in the cutting configuration than in the retracted configuration, and wherein the outer blade and the at least one inner blade cooperatively define a tapered cutting profile; advancing the at least one inner blade to the cutting configuration; and reciprocating the at least one inner blade proximally and distally or rotating the telescoping cutting assembly to cut a lesion at the intervention site.

In yet another embodiment, a system for atherectomy or thrombectomy procedure is provided. The system may include: an intravascular cutting tool, including: a catheter body defining an aspiration lumen extending through the catheter body, and a telescoping cutting assembly including an outer blade coupled to and extending distally from the catheter body and at least one inner blade positioned radially inward of the outer blade, the at least one inner blade being axially moveable between a retracted configuration and a cutting configuration, wherein the at least one inner blade extends more distally beyond the outer blade when in the cutting configuration than in the retracted configuration, and wherein the outer blade and the at least one inner blade cooperatively define a tapered cutting profile; and an aspiration stylet advanceable through the aspiration lumen and an opening through the telescoping cutting assembly.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 is an isometric view of an intravascular cutting tool, according to one or more embodiments shown and described herein;

FIG. 2 is a cross-sectional side view of the intravascular cutting tool of FIG. 1 positioned in an at least partially retracted configuration, according to one or more embodiments shown and described herein;

FIG. 3 depicts the intravascular cutting tool of FIGS. 1-2 positioned in a cutting configuration, according to one or more embodiments shown and described herein;

FIG. 4 illustrates example operation of the intravascular cutting tool of FIGS. 1-3 during an occlusion removal procedure wherein the intravascular cutting tool includes an aspiration stylet, according to one or more embodiments shown and described herein; and

FIG. 5 is an end view of an alternate intravascular cutting tool having semi-circular blades, according to one or more embodiments shown and described herein.

Reference will now be made in greater detail to various embodiments of the present disclosure, some embodiments of which are illustrated in the accompanying drawings. Whenever possible, the same reference numerals will be used throughout the drawings to refer to the same or similar parts.

### DETAILED DESCRIPTION

Embodiments described herein are directed to an intravascular cutting tool useful in removing occlusions from a vessel (e.g., a blood vessel or other bodily vessel). Occlusion removal procedures may include, but are not limited to atherectomy and thrombectomy procedures.

Embodiments of the intravascular cutting tool generally includes a catheter body and a telescoping cutting assembly. The cutting assembly generally includes an outer blade coupled to and extending distally from the catheter body and at least one inner blade positioned radially inward of the outer blade. The at least one inner blade is axially moveable between a retracted configuration and a cutting configuration where the at least one inner blade extends distally beyond the outer blade when in the cutting configuration than in the retracted configuration. The outer blade and the at least one inner blade cooperatively define a tapered cutting profile allowing it to be more easily traversed through a subject's vasculature. The intravascular cutting tool is able to cut and remove occlusions (e.g., atheromatous plaque or thrombi) from within a vessel (e.g., an artery or vein) via axial movement and/or rotational movement. In embodiments, the intravascular cutting tool may remove occlusive material without cutting the endothelial layer of the vessel. Various embodiments of the intravascular cutting tool and the operation of the intravascular cutting tool are described in more detail herein. Whenever possible, the same reference numerals will be used throughout the drawings to refer to the same or like parts.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Directional terms as used herein-for example up, down, right, left, front, back, top, bottom-are made only with reference to the figures as drawn and are not intended to imply absolute orientation. The terms “proximal” and “distal” are defined herein relative to an origin of a device such as, for example a handle of a catheter. The term “proximal” refers to the position of an element closer to the origin of the catheter and the term “distal” refers to the position of an element further away from the origin such as toward the tip of the catheter.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

As used herein, the singular forms “a,” “an” and “the” include plural referents unless the context clearly dictates otherwise. Thus, for example, reference to “a” component includes aspects having two or more such components, unless the context clearly indicates otherwise.

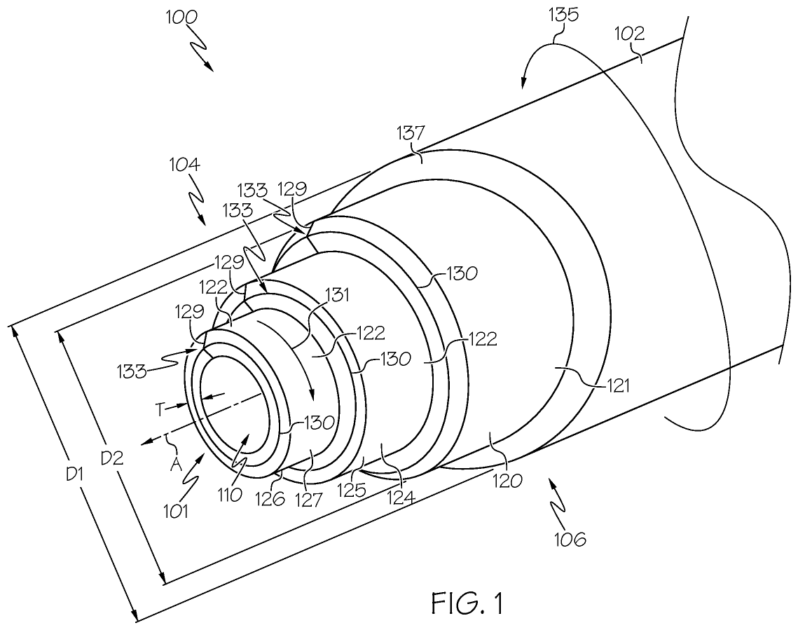

Referring now to FIG. 1, an intravascular cutting tool 100 is illustrated according to one or more embodiments described herein. The intravascular cutting tool 100 extends longitudinally along an axis A between a distal end 101 and a proximal end (not shown). The intravascular cutting tool 100 may generally include a catheter 102 and a telescoping cutting assembly 104 provided at a distal end 106 of the catheter 102. The catheter 102 comprises a catheter body that longitudinally extends from the distal end 106 to a proximal end (not illustrated). A handle (not shown) may be provided at the proximal end, and an actuator (not shown) may be provided in the handle or at the proximal end for controlling movement of the telescoping cutting assembly 104 as described below. The catheter 102 may be formed of any conventional materials for traversing through a subject's vasculature. For example, the catheter 102 may be formed polyurethane, silicon, or the like. As depicted, the telescoping cutting assembly 104 is mounted to the distal end 106 of the catheter 102.

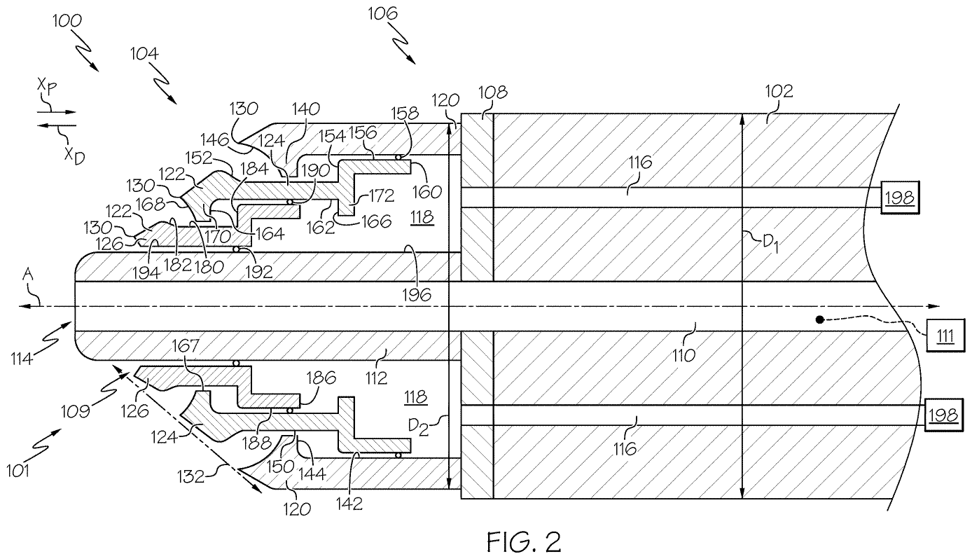

FIG. 2 is a cross-sectional view of the intravascular cutting tool 100 of FIG. 1. In the illustrated embodiment, the telescoping cutting assembly 104 is mounted to the distal end 106 of the catheter 102 via a rotational bearing 108. In embodiments, the rotational bearing 108 is an motorized rotary stage electric motor that it is operable to impart rotation to the telescoping cutting assembly 104 upon actuation. For example, the rotational bearing 108 may include a micromotor and one or more gearings to provide a desired amount of torque, wherein the micromotor causes rotation of the telescoping cutting assembly 104. In embodiments, the rotational bearing 108 is configured as an air motor that imparts rotation to the telescoping cutting assembly 104 upon application of compressed air thereto, for example, the rotational bearing 108 may include an air turbine that rotates about the axis A when air is applied thereon, thereby driving rotation of the telescoping cutting assembly 104. In other, non-illustrated embodiments, the rotational bearing 108 is not utilized and the telescoping cutting assembly 104 is instead attached directly to the distal end 106, wherein rotation is imparted on the telescoping cutting assembly 104 via rotating the catheter 102.

In embodiments, the telescoping cutting assembly 104 is integral with the rotational bearing 108, or otherwise coupled to the rotational bearing 108 such that the combination of the telescoping cutting assembly 104 and the rotational bearing 108 is removable from the catheter 102 such that the user may exchange the telescoping cutting assembly 104, for example, to change cutting diameter of the telescoping cutting assembly 104 and/or to change blade type of the telescoping cutting assembly 104, etc. For example, the rotational bearing 108 may have a externally threaded surface that meshes within a threaded surface in a lumen of the catheter 102, or the rotational bearing 108 may be friction fit within the lumen of the catheter 102. In other embodiments, the rotational bearing 108 is affixed to the distal end 106 of the catheter 102 and the telescoping cutting assembly 104 is removable from the combination of the catheter 102 and the rotational bearing 108. For example, the telescoping cutting assembly 104 may include a threaded surface that messes with a corresponding threaded surface of the rotational bearing 108, or the rotational bearing 108 and the telescoping cutting assembly 104 may be friction fit together. In other embodiments, the telescoping cutting assembly 104 is removable directly from the distal end 106 of the catheter 102, such that there is no rotational bearing.

The catheter 102 has an outer diameter D1. The outer diameter of the D1 may be selected based on the vessel within which the catheter 102 is to be inserted during a procedure. Accordingly, the outer diameter D1 of the catheter 102 may be selected based on the particular procedure and subject. For example, the catheter 102 may be selected such that the outer diameter D1 thereof closely approximates but is slightly smaller than an inner diameter of a vessel (e.g., artery or vein) within which it is to be inserted during a procedure.

The catheter 102 defines at least part of a lumen 110 extending therethrough. As shown, the lumen 110 extends longitudinally through the body of the catheter 102, such as from the proximal end to the distal end 106. As hereinafter described, the lumen 110 of the catheter 102 may provide an aspiration pathway for extracting pieces of occlusive material (e.g., atheromatous plaque, thrombi, clots) that have been dislodged or cut-away from within a vessel via the intravascular cutting tool 100. For example, an aspiration device 111 (e.g., a vacuum pump or the like) may be coupled in fluid communication with the lumen 110 such that, upon activation of the aspiration device 111, suction is applied through the lumen 110 to draw or pull particles proximally through the lumen 110. For example, particles of occlusive material (e.g., atheromatous plaque, clot, thrombus) that have been dislodged or broken off via the telescoping cutting assembly 104 may be sucked into the lumen 110 and extracted from the subject's vessel.

In the illustrated embodiment, an aspiration tip 112 is disposed at the distal end 106 of the catheter 102, and the aspiration tip 112 extends distally through an opening 109 defined within the telescoping cutting assembly 104. The aspiration tip 112 may be a tube or cannula made from of a similar material as the catheter 102, in some embodiments, the aspiration tip 112 may be formed of a more rigid material than the catheter 102. Accordingly, the aspiration tip 112 may define a portion of the lumen 110, such that the lumen 110 extends distally beyond the distal end 106 of the catheter 102, into and through the rotational bearing 108, and into and through the aspiration tip 112. In embodiments, the lumen 110 terminates at an opening 114 defined by the aspiration tip 112.

Also, at least one pressure lumen 116 may be provided within the catheter 102 and extends through the rotational bearing 108 (and/or the distal end 106 of the catheter 102) so as to communicate with a cavity 118 defined within the telescoping cutting assembly 104. As hereinafter described, the at least one pressure lumen 116 may be utilized to apply positive or negative pressure within the cavity 118 to actuate the telescoping cutting assembly 104.

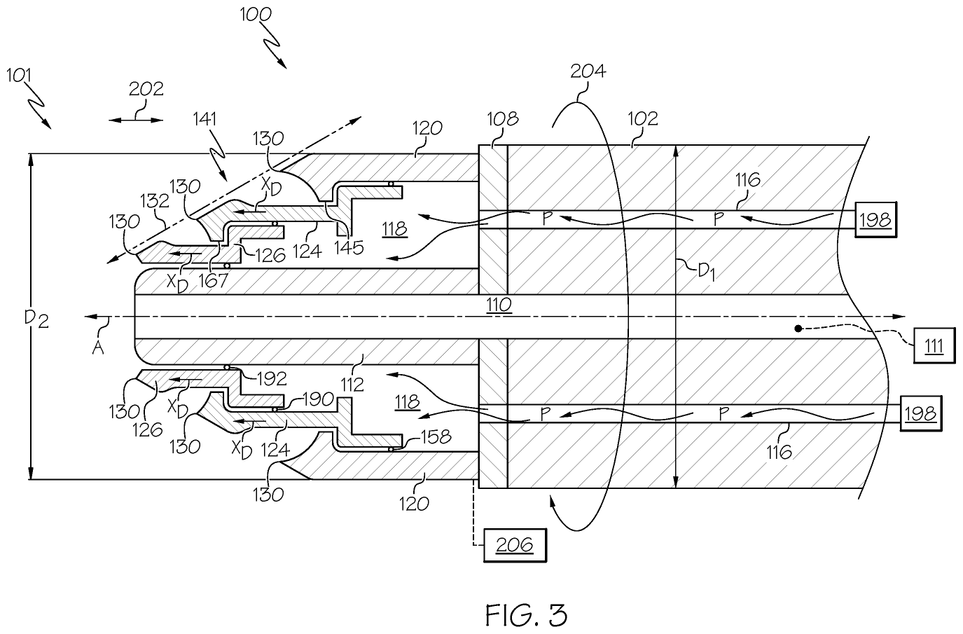

The telescoping cutting assembly 104 generally includes an outer blade 120 and at least one inner blade 122. As hereinafter described, the at least one inner blade 122 is movable between a retracted configuration and a cutting configuration. FIG. 2 is a cross-sectional view of the intravascular cutting tool 100 of FIG. 1 depicting the at least one inner blade 122 and FIG. 3 is a cross-sectional view of the intravascular cutting tool 100 of FIGS. 1-2 wherein the at least one inner blade 122 has been moved into the cutting configuration.

In embodiments, the outer blade 120 and the at least one inner blade 122 are each circular, and thus extend continuously about a circumference of the intravascular cutting tool 100. However, either or both of the outer blade 120 and the at least one inner blade 122 may have different geometries. For example, either or both of the outer blade 120 and the at least one inner blade 122 may be semi-circular, such that they have a gap in their outer circumference such that they do not extend continuously about a circumference of the intravascular cutting tool 100. In embodiments, the outer blade 120 and the at least one inner blade 122 may be configured as a milling cutting tool. In embodiments, the outer blade 120 and the at least one inner blade 122 may be configured as a drilling cutting tool.

For example, FIG. 1 illustrates an exemplary embodiment of the distal end 101 of the intravascular cutting tool 100 wherein the outer blade 120, the first inner blade 124, and the second inner blade 126 are each provided as a circular ring or cylinder, according to one or more embodiments shown and described herein. As shown, the outer blade 120 may be provided in an outer cylinder (or outer ring) 121, the first inner blade 124 may be provided in a first inner cylinder (or a first inner ring) 125, and the second inner blade 126 may be provided in a second inner cylinder (or a second inner ring) 127 that is positioned radially inward of the first inner cylinder 404. In embodiments, one or more of the cutting edges 130 of the respective blades may extend continuously around each of the outer cylinder 121, the first inner cylinder 125, and the second inner cylinder 127 (collectively, “the cylinders 121,125,127”).

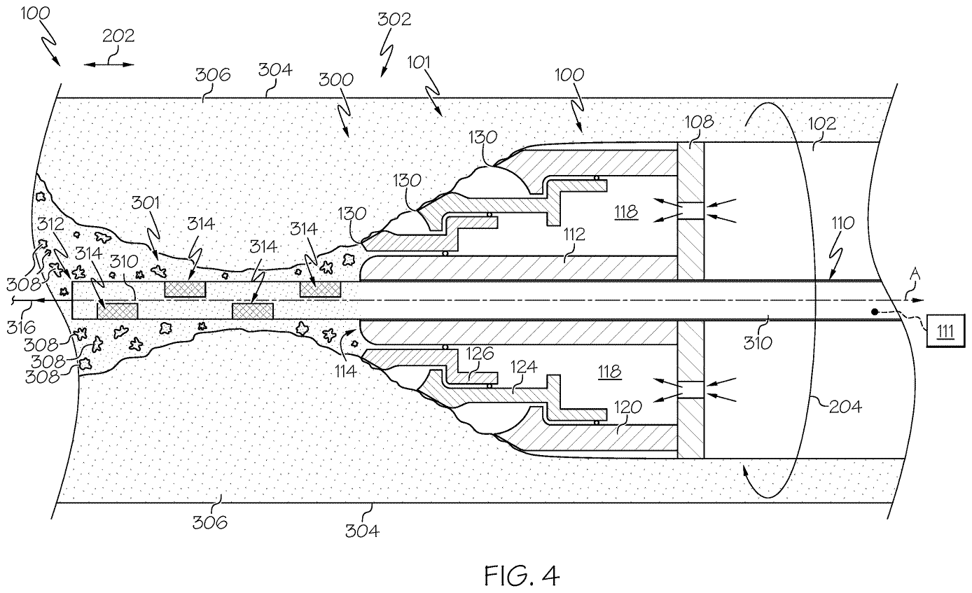

However, in other embodiments, any one or more of the cutting edge 130 may be discontinuous and/or may exhibit a partially spiral geometry that begins a first point, extends around the respective cylinder, and ends at a second point that is distal or proximal from the first point. For example, FIG. 1 illustrates an embodiment where each of the cutting edges 130 begins at an edge 129 and then extends around its respective one of the cylinders 121, 125,127 as indicated by an arrow 131, and then terminates at an exposed sidewall 133 at a location thereon that is proximal of the edge 129, such that the edge 129 is exposed. The edge 129 on each of the cylinders 121,125,127 is defined by the exposed sidewall 133 associated therewith and has a width dimension that is equal to a thickness T of the particular one of the cylinders 121,125,127 associated therewith. The exposed sidewall 133 also has a width dimension that is equal to the thickness T associated therewith, and also has a length dimension measured along the axis A between a starting point of the cutting edge 130 at the edge 129 and the ending point of the cutting edge 130 at the exposed sidewall 133. When rotated as indicated by arrow 135, the exposed sidewall 133 edge the edge 129 will abrade and scrape against a blockage (e.g., as shown in FIG. 4). In some embodiments, a cutting edge may be formed on the edge 129 so as to cut or slice the blockage as telescoping cutting assembly 104 is rotated as indicated by the arrow 135.

The catheter 102 may include a tapered edge 137 which tapers in the distal direction. With the tapered edge 137, the intravascular cutting tool 100 is able to gradually reduce its outer diameter, for example, from the outer diameter D1 of the catheter 102 to the maximum outer diameter D2 of the outer cylinder 121, thereby facilitating access within the arteries or vessels. However, the tapered edge 137 is optional and, in some embodiments, the tapered edge 137 of the catheter 102 is instead formed as a distally facing end face that is substantially perpendicular to an outer circumferential surface of the outer cylinder 121. Regardless, the intravascular cutting tool 100 is effectively self-centering within the vessel without necessary adjustment by the user via sizing of the outer diameter D1 of the catheter 102. For example, by selecting the catheter 102 with the outer diameter D1 that closely approximates the inner diameter of the vessel within which the intravascular cutting tool 100 is to be inserted, the telescoping cutting assembly 104 will be immediately centered inside the vessel without further adjustment by the user. The outer diameter D1 may be selected based on the size (i.e., the inner diameter) of the artery or vein within which the intravascular cutting tool 100 is to be inserted. Also, providing the telescoping cutting assembly 104 with the maximum outer diameter D2 that is smaller than the outer diameter D1 of the catheter 102 helps ensure that the telescoping cutting assembly 104 does not accidentally contact or cut the inner walls of the vessel as it navigates to and from an intervention site (e.g., as shown in FIG. 4).

As shown in FIG. 2, the outer blade 120 is coupled to the distal end 106 of the catheter 102 and extends distally therefrom. In the illustrated embodiment, the outer blade 120 is attached to the rotational bearing 108. In this manner, the outer blade 120 may rotate relative to the catheter 102. However, as mentioned above, rotation of the outer blade 120 may be caused via rotation of the catheter 102 in embodiments without the rotational bearing 108.

The at least one inner blade 122 is nested within the outer blade 120. In some embodiments, the at least one inner blade 122 comprises a plurality of inner blades and, as more fully described below, the most radially outward positioned inner blade is nested within the outer blade 120, and the next inner blade positioned radially inward from the most radially outward positioned inner blade is nested within the most radially outward positioned inner blade, and so on.

In the illustrated embodiment, the at least one inner blade 122 includes a first inner blade 124 and a second inner blade 126 that is positioned radially inward of the first inner blade 124. Stated differently, the first inner blade 124 is positioned radially outward of the second inner blade 126. As hereinafter described, the first inner blade 124 and the second inner blade 126 are axially movable along the axis A, relative to each other and/or to the outer blade 120.

A cutting edge 130 is formed on each of the outer blade 120, the first inner blade 124, and the second inner blade 126. When the first inner blade 124 and the second inner blade 126 are moved proximally, relative to the outer blade 120, into the retracted configuration, the orientation of the cutting edges 130 together (or cooperatively) define a tapered cutting profile 132 exhibiting a maximum outer diameter D2 that is less than the outer diameter D1 of the catheter 102, as shown in FIG. 2. When in the retracted configuration, the tapered cutting profile 132 cooperatively defined by the cutting edges 130 of the telescoping cutting assembly 104 relative to the distal tip 114 inhibits the sharp edges from contacting interior walls of the arteries or vessels when advancing or retracting the intravascular cutting tool 100 to or from the intervention site. Also, when in the retracted configuration, the aspiration tip 112 may extend distally beyond each of the outer blade 120, the first inner blade 124, and the second inner blade 126, which further inhibits the sharp edges from contacting interior walls of the arteries or vessels when advancing or retracting the intravascular cutting tool 100 to or from the intervention site. In the illustrated embodiment, the cutting edge 130 of the outer blade 120 defines an outer cutting perimeter of the telescoping cutting assembly 104, and the cutting edges 130 of the first inner blade 124 and the second inner blade 126 are positioned within (or interior of or radially inward of) the outer cutting perimeter.

The outer blade 120 may include a retention feature 140 that regulates axial translation of the first inner blade 124 (i.e., inhibits axial translation of the first inner blade 124 beyond a most distal position). In the illustrated example, the retention feature 140 is provided at a distal end 141 of the outer blade 120. Here, the retention feature 140 extends radially inward from an inward facing surface 142 of the outer blade 120, towards the at least one inner blade 122, to thereby define a proximally facing stop surface 144, a radially inward facing slide surface 145, and a distally oriented stop surface 146. The retention feature 140 may take any suitable shape configured to inhibit axial translation of the first inner blade 124 beyond the most distal position by abutting the first inner blade 124 in the most distal position.

The first inner blade 124 may include a radially outward facing slide surface 150 which engages and slides upon the radially inward facing slide surface 145 of the retention feature 140 of the outer blade 120. The first inner blade 124 also includes a proximally oriented stop surface 152 that may abut the distally oriented stop surface 146 of the retention feature 140 of the outer blade 120 when the first inner blade 124 fully translates (or telescopes) in a proximal direction X_{P} such that the first inner blade 124 is in the retracted configuration. The proximally oriented stop surface 152 may have any suitable shape. Thus, as the first inner blade 124 translates in the proximal direction X_{P}, the proximally oriented stop surface 152 of the first inner blade 124 may eventually contact the distally oriented stop surface 146 of the retention feature 140 of the outer blade 120 thereby stopping any further proximal translation of the first inner blade 124. In addition, the first inner blade 124 includes a distally facing stop surface 154 arranged to abut the proximally facing stop surface 144 of the retention feature 140 of the outer blade 120 when the first inner blade 124 fully translates (or telescopes) in a distal direction X_{P}such that the first inner blade 124 is extended into the cutting configuration. The distally facing stop surface 154 may have any suitable shape. Thus, as the first inner blade 124 translates in the distal direction X_{P}, the distally facing stop surface 154 of the first inner blade 124 may eventually contact the proximally facing stop surface 144 of the retention feature 140 of the outer blade 120 thereby stopping any further distal translation of the first inner blade 124. Accordingly, the retention feature 140 of the outer blade 120 limits or regulates axial translation or motion of the first inner blade 124.

The first inner blade 124 may also include an outward facing engagement surface 156 that extends proximally from the distally facing stop surface 154 and is oriented to face the inward facing surface 142 of the outer blade 120. In the illustrated example, a seal or gasket 158 is optionally disposed between the outward facing engagement surface 156 of the first inner blade 124 and the inward facing surface 142 of the outer blade 120, so as to inhibit fluid flow (e.g., liquid or air) there through. In embodiments, the gasket 158 may be an O-ring seal set within a recess (e.g., a slot or groove) formed in the outward facing engagement surface 156, and extending circumferentially around the first inner blade 124, such that the gasket 158 slides on the inward facing surface 142 of the outer blade 120, with the first inner blade 124 and relative to the outer blade 120. As hereinafter described, the gasket 158 may facilitate pressurization of the cavity 118 to cause translation of the first inner blade 124 and the second inner blade 126.

In some embodiments, the first inner blade 124 is sized to be sufficiently long enough that a proximal end 160 of the first inner blade 124 abuts or contacts the distal end 106 of the catheter or a distal face of the rotational bearing 108 when the first inner blade 124 is fully translated (or collapsed) proximally into the retracted configuration. For example, the outward facing engagement surface 156 may be long enough such that, when the first inner blade 124 is in the retracted configuration, the proximal end 160 of the first inner blade 124 abuts or contacts the distal end 106 of the catheter or a distal face of the rotational bearing 108. In embodiments, when the first inner blade 124 is in the retracted configuration, the proximal end 160 of the first inner blade 124 abuts the distal face of the rotational bearing 108 (or the distal end 106 of the catheter 102) and the proximally oriented stop surface 152 of the first inner blade 124 also contacts its corresponding surface of the outer blade 120. However, in other embodiments, when the first inner blade 124 is in the retracted configuration, the proximal end 160 of the first inner blade 124 abuts the distal face of the rotational bearing 108 (or the distal end 106 of the catheter 102) or the proximally oriented stop surface 152 of the first inner blade 124 contacts its corresponding surface of the outer blade 120.

The first inner blade 124 may include an inward facing surface 162. The first inner blade 124 may also include a proximally facing stop surface 164 and a distally facing stop surface 166, wherein the proximally facing stop surface 164 is disposed at a distal end of the inward facing surface 162 and the distally facing stop surface 166 is disposed at a proximal end of the inward facing surface 162. In the illustrated embodiment, the second inner blade 126 slides against the inward facing surface 162, between the proximally facing stop surface 164 and the distally facing stop surface 166, such that a length of the inward facing surface 162 defines the amount of travel that the second inner blade 126 may exhibit as it slides between its cutting configuration and retracted configuration. The stop surfaces 164, 166 may take any suitable shape for inhibiting axial movement of the second inner blade 126 in the proximal and distal direction, respectively. Further, the first inner blade 124 may include a distally oriented stop surface 168 and a radially inward facing slide surface 167 extending between the distally oriented stop surface 168 and the proximally facing stop surface 164, each of which may similarly have any suitable shape. In the illustrated embodiment, the proximally facing stop surface 164, the radially inward facing slide surface 167, and the distally oriented stop surface 168 define a retention feature 170 of the first inner blade 124 that regulates the extent of distal translation that the second inner blade 126 may travel, whereas the distally facing stop surface 166 defines a proximal feature 172 of the first inner blade 124 that regulates the extent of proximal translation that the second inner blade 126 may travel. Also, in some embodiments, the distally oriented stop surface 168 may also regulate the extent of proximal translation that the second inner blade 126 may travel.

The second inner blade 126 may include a radially outward facing slide surface 180 which engages and slides upon the radially inward facing slide surface 167 of the retention feature 170 of the first inner blade 124. The second inner blade 126 may also include a proximally oriented stop surface 182 that may abut the distally oriented stop surface 168 of the retention feature 170 of the first inner blade 124 when the second inner blade 126 fully translates (or collapses) in the proximal direction X_{P} such that the second inner blade 126 is in the retracted configuration. Thus, as the second inner blade 126 translates in the proximal direction X_{P}, the proximally oriented stop surface 182 of the second inner blade 126 may eventually contact the distally oriented stop surface 168 of the retention feature 170 of the first inner blade 124 thereby stopping any further proximal translation of the second inner blade 126. In the illustrated embodiment, the second inner blade 126 may include a proximally facing stop surface 186 arranged at a proximal end of second inner blade 126, wherein the proximally facing stop surface 186 abuts the distally facing stop surface 166 of the proximal feature 172 of the first inner blade 124 when the second inner blade 126 fully translates (or collapses) in the proximal direction X_{P} into the retracted configuration. Thus, as the second inner blade 126 translates in the proximal direction X_{P}, the proximally facing stop surface 186 of the second inner blade 126 may eventually contact the distally facing stop surface 166 of the proximal feature 172 of the first inner blade 124 thereby stopping any further proximal translation of the second inner blade 126 relative to the first inner blade 124. In embodiments, both the proximally oriented stop surface 182 and the proximally facing stop surface 186 contact their corresponding surfaces of the first inner blade 124 when in the retracted configuration; however, in other embodiments just the proximally oriented stop surface 182 or just the proximally facing stop surface 186 contacts its corresponding surface of the first inner blade 124 when in the retracted configuration.

In addition, the second inner blade 126 may include a distally facing stop surface 184 arranged to abut the proximally facing stop surface 164 of the retention feature 170 of the first inner blade 124 when the second inner blade 126 fully translates (or telescopes) in the distal direction X_{D} such that the second inner blade 126 is extended into the cutting configuration. Thus, as the second inner blade 126 translates in the distal direction X_{P}, the distally facing stop surface 184 of the second inner blade 126 may eventually contact the proximally facing stop surface 164 of the retention feature 170 of the first inner blade 124 thereby stopping any further distal translation of the second inner blade 126. Accordingly, the first inner blade 124 regulates axial translation of the second inner blade 126.

The second inner blade 126 may include an outward facing engagement surface 188 that extends between the distally facing stop surface 184 and the proximally facing stop surface 186 and is oriented to face the inward facing surface 162 of the first inner blade 124. In the illustrated example, a seal or gasket 190 is disposed between the outward facing engagement surface 188 of the second inner blade 126 and the inward facing surface 162 of the first inner blade 124, so as to inhibit fluid flow (e.g., liquid or air) there through. In embodiments, the gasket 190 may be an O-ring seal that is set within a recess (e.g., a slot or groove) formed in the outward facing engagement surface 188, and extending circumferentially around the second inner blade 126, such that the gasket 190 slides on the inward facing surface 162 of the first inner blade 124, with the second inner blade 126 and relative to the first inner blade 124. As hereinafter described, the gasket 190 may facilitate pressurization of the cavity 118 to cause translation of the first inner blade 124 and the second inner blade 126.

As previously mentioned, the first inner blade 124 may be sealed against the outer blade 120 via the gasket 158 and the second inner blade 126 may be sealed against the first inner blade 124 via the gasket 190. In the illustrated embodiment, the second inner blade 126 may be sealed against the aspiration tip 112 via a seal or gasket 192. Here, the gasket 192 is disposed between an outward facing engagement surface 194 of the second inner blade 126 and a surface 196 of the aspiration tip 112, so as to inhibit fluid flow (e.g., liquid or air) there through. In embodiments, the gasket 192 may be an O-ring seal that is set within a recess (e.g., slot or groove) formed in the outward facing engagement surface 194, and extending circumferentially around the second inner blade 126, such that the gasket 192 slides on the surface of the aspiration tip 112, with the second inner blade 126 and relative to the aspiration tip 112. As hereinafter described, the gasket 192 may facilitate pressurization of the cavity 118 to cause translation of the first inner blade 124 and the second inner blade 126.

As shown, the cavity 118 may be defined between the outer blade 120, the at least one inner blade 122, the rotational bearing 108 (or the distal end 106 of the catheter 102), and the aspiration tip 112. In the illustrated example, the cavity 118 is annular in shape due to cylindrical shape of the outer blade 120, the first inner blade 124, the second inner blade 126, and the aspiration tip 112 within which the cavity 118 is constrained. By incorporating the gaskets 158, 190, 192, positive or negative pressure may be applied to the cavity 118, via the at least one pressure lumen 116, to extend the first inner blade 124 and the second inner blade 126 into the cutting configuration or to retract them into the retracted configuration, respectively. In addition, the gaskets 158, 190, 192 may substantially seal the cavity 118 from an ambient (external) environment such that external fluids (e.g., blood) may not enter the cavity 118 during use.

In embodiments, the intravascular cutting tool 100 includes a pressure source 198 for pressurizing the cavity 118 and controlling linear displacement of the first inner blade 124 and the second inner blade 126. The pressure source 198 may be in fluid communication with the at least one pressure lumen 116, such that the pressure source 198 may apply positive or negative pressure P to the cavity 118 through the at least one pressure lumen 116. In embodiments, the pressure source 198 may be configured to apply hydraulic pressure; whereas in other embodiments, the pressure source 198 may be configured to apply pneumatic pressure. In embodiments, the pressure source 198 is a pump that is fluidically coupled to the at least one pressure lumen 116, for example, via a tube or conduit. Thus, the pressure source 198 may be provided exterior the catheter 102, for example, exterior the proximal end of the catheter 102 such that the surgeon may control pressurization of the cavity 118 while separately manipulating the proximal end of the catheter 102. In embodiments, the rotational bearing 108 is configured as an air turbine and is driven by compressed air delivered by the at least one pressure lumen 116.

In embodiments, the telescoping cutting assembly 104 may, instead of or in addition to pressurized actuation, be mechanically actuated. For example, the first inner blade 124 and/or the second inner blade 126 may each include a drive rod (not shown) that extends proximally through the catheter 102, wherein the drive rods are each connected to a motor (not illustrated) that distally and proximally reciprocates the first inner blade 124 and the second inner blade 126 between the cutting configuration and the retracted configuration. In embodiments, the telescoping cutting assembly 104 may be actuated via inertia resulting from rotation of the telescoping cutting assembly 104. For example, rotation of the telescoping cutting assembly 104 may in a first rotational direction may cause the first inner blade 124 and/or the second inner blade 126 to move distally into the cutting configuration and rotation in the opposite direction may cause the first inner blade 124 and/or the second inner blade 126 to move proximally into the retracted configuration. Thus, when the telescoping cutting assembly 104 is rotated in the same direction, the first inner blade 124 and/or the second inner blade 126 may be maintained in the cutting configuration. Alternatively, the telescoping cutting assembly 104 may be rotated in a first rotational direction such that the first inner blade 124 and/or the second inner blade 126 move distally into the cutting configuration and then the telescoping cutting assembly 104 may be rotated in an opposite direction such that the first inner blade 124 and/or the second inner blade 126 move proximally into the retracted configuration, and such back and forth rotation of the telescoping cutting assembly 104 may be continued over time such that the first inner blade 124 and/or the second inner blade 126 continuously move distally and then proximally between the cutting configuration and the retracted configuration, similar to a hammering type movement.

Embodiments of the telescoping cutting assembly 104 provided herein allow for cutting through a completely or partially occluded or calcified vessel or artery, as it is not necessary to extend a guide wire through the occlusion in order to drive the telescoping cutting assembly 104 there through. Further, the telescoping cutting assembly 104 allows the first inner blade 124 and the second inner blade 126 to advance and accommodate to the specific shape of the lesion and occlusion size without having to change tool sizes.

In embodiments, the pressure source 198 comprises a hand held device (not shown), wherein a positive pressure is applied to the cavity 118 via actuation of a trigger of the hand held device. In some embodiments, the hand held device includes a separate trigger for applying negative pressure that causes retraction of the first inner blade 124 and the second inner blade 126. In some embodiments, the hand held device is configured to apply negative pressure within the cavity 118 when the trigger is not actuated, such that the telescoping cutting assembly 104 defaults to the retracted configuration when the trigger is not engaged (e.g., pulled); and then pulling the trigger causes the hand held device to apply positive pressure within the cavity 118 to thereby extend the telescoping cutting assembly 104 into the cutting configuration, and releasing the trigger causes the hand held device to default back to application of negative pressure within the cavity 118 to retract the telescoping cutting assembly 104. In embodiments, the trigger of the hand held device is operable to activate and regulate delivery of pressure to the telescoping cutting assembly 104 to thereby extend and retract the first inner blade 124 and the second inner blade 126. In some embodiments, a biasing element (e.g., a spring) may be utilized to bias the first inner blade 124 and/or the second inner blade 126 into the retracted configuration, and the pressure source 198 is configured to provide sufficient positive pressure to overcome such biasing force. In some embodiments, the trigger of the hand held device is also operable to activate and regulate delivery of pressure to the telescoping cutting assembly 104 to thereby cause rotation of the telescoping cutting assembly 104.

In FIG. 2, the first inner blade 124 and the second inner blade 126 are depicted in an at least partially retracted configuration, wherein the first inner blade 124 and the second inner blade 126 have been at least partially translated in the proximal direction X_{P}. Here, neither the proximally oriented stop surface 152 nor the proximal end 160 of the first inner blade 124 is depicted as being in contact with the distally oriented stop surface 146 of the outer blade 120 or the distal face of the rotational bearing 108, respectively, but the first inner blade 124 is at least partially translated in the proximal direction X_{P} towards the retracted configuration. However, it will be appreciated that the first inner blade 124 may be fully translated in the proximal direction X_{P} such that, when in the retracted configuration, the proximally oriented stop surface 152 abuts the distally oriented stop surface 146 of the outer blade 120 and/or that the proximal end 160 of the first inner blade 124 abuts the distal face of the rotational bearing 108.

Similarly, neither the proximally oriented stop surface 182 nor the proximally facing stop surface 186 of the second inner blade 126 is depicted as being in contact with the distally oriented stop surface 168 of the first inner blade 124 or the distally facing stop surface 166 of the first inner blade 124, respectively, but the second inner blade 126 is at least partially translated in the proximal direction X_{P} towards the retracted configuration. However, it will be appreciated that the second inner blade 126 may be fully translated in the proximal direction X_{P} such that, when in the retracted configuration, the proximally oriented stop surface 182 abuts the distally oriented stop surface 168 of the first inner blade 124 and/or that the proximally facing stop surface 186 of the second inner blade 126 abuts the distally facing stop surface 166 of the first inner blade 124.

As mentioned above, FIG. 3 illustrates the intravascular cutting tool 100 wherein the telescoping cutting assembly 104 is in the cutting configuration. In the illustrated embodiment, the pressure source 198 applies the pressure P through the at least one pressure lumen 116 to thereby pressurize the cavity 118. When the pressure P is a positive pressure, for example, in the form of compressed air or fluid forced into the cavity 118, the pressure P exerts a force on the first inner blade 124 and the second inner blade 126 which drives the first inner blade 124 and the second inner blade 126 in the distal direction X_{P} and into the cutting configuration. Such distal translation of the first inner blade 124 is limited by the proximally facing stop surface 144 of the outer blade 120 which, when contacted by the distally facing stop surface 154 of the first inner blade 124, inhibits any further travel in the distal direction XD. Similarly, such distal translation of the second inner blade 126 is limited by the proximally facing stop surface 164 of the first inner blade 124 which, when contacted by the distally facing stop surface 184 of the second inner blade 126, inhibits any further travel of the second inner blade 126 in the distal direction XD. Accordingly, the cutting edges 130 formed on the first inner blade 124 and the second inner blade 126 extend further distally from the cutting edge 130 on the outer blade 120 when in the cutting configuration than when in the retracted configuration.

The pressure source 198 may also apply negative pressure within the cavity 118 to at least partially retract the telescoping cutting assembly 104 such that the first inner blade 124 and the second inner blade 126 at least partially move in the proximal direction X_{P}. Such negative pressure may be utilized to fully retract the first inner blade 124 and the second inner blade 126 into the retracted configuration, after which the intravascular cutting tool 100 may be extracted from the subject. In embodiments, the pressure source 198 may be configured to continuously alternate between supplying positive and negative within the cavity 118, such that the first inner blade 124 and the second inner blade 126 are continuously moving back and forth between the distal direction X_{D} and the proximal direction X_{P}, as indicated by arrow 202. Thus, during an atherectomy or thrombectomy procedure, the pressure source 198 may be configured to cause the first inner blade 124 and the second inner blade 126 to longitudinally oscillate or reciprocate back and forth as indicated by the arrow 202, such that the first inner blade 124 and the second inner blade 126 continuously “stab” or “chop” the occlusive material (atheromatous plaque, thrombi, or clot) from within the vessel, and the aspiration device 111 may provide negative pressure within the lumen 110 to suck or draw any dislodges the released occlusive material proximally through the lumen 110 and out of the vessel.

Also during an atherectomy or thrombectomy procedure, the telescoping cutting assembly 104 may be rotated as indicated by an arrow 204, as shown in FIGS. 3-4. While the arrow 204 illustrates a rotation in a first rotational direction (e.g., clockwise), the telescoping cutting assembly 104 may instead be rotated in a second rotational direction that is opposite to the first rotational direction (e.g., counter-clockwise) as indicated by the arrow 135 in FIG. 1. As shown in FIG. 3, a motor 206 may be operatively coupled to the telescoping cutting assembly 104 and drives rotation of the telescoping cutting assembly 104. In embodiments, the motor 206 is integrated within the rotational bearing 108 and operable to cause rotation of a portion of the rotational bearing 108 that is attached to the telescoping cutting assembly 104. In embodiments, the motor 206 is integrated within the catheter 102, for example, at the distal end 106 of the catheter 102 proximate to the rotational bearing 108, and operable to cause rotation of a portion of the rotational bearing 108 that is attached to the telescoping cutting assembly 104. In embodiments, the motor 206 is integrated in the telescoping cutting assembly 104, for example, within the cavity 118 defined therein. In embodiments, rotation imparted on the telescoping cutting assembly 104 via the motor 206 may create inertia that is utilized to deploy or retract the first inner blade 124 and/or the second inner blade 126, as mentioned above, such that they may continuously reciprocate between the cutting and retracted configurations as the rotational direction is continuously reversed, or such that they are maintained in either configuration as the telescoping cutting assembly 104 is rotated in a single rotational direction. As previously mentioned, the telescoping cutting assembly 104 may be coupled to the distal end 106 of the catheter 102 via the rotational bearing 108, and the rotational bearing 108 permits the telescoping cutting assembly 104 to rotate relative to the catheter 102. In other embodiments, the telescoping cutting assembly 104 may be provided directly on the catheter 102, such that rotation is imparted on the telescoping cutting assembly 104 by rotating the catheter 102 (e.g., by rotating the proximal end of the catheter 102). When rotating, the cutting edges 130 of the telescoping cutting assembly 104 operate to “slice” the occlusive material from within the vessel, and the aspiration device 111 may provide negative pressure within the lumen 110 to suck or draw any dislodged occlusive material proximally through the lumen 110 and out of the vessel.

In embodiments, the motor 206 is configured to alternate rotation direction of the telescoping cutting assembly 104. For example, the motor 206 may rotate the telescoping cutting assembly 104 as indicated by the arrow 204 for a certain period of time or for a certain rotational distance, and then the motor 206 may reverse rotational direction of the telescoping cutting assembly 104 such that it rotates in an opposite direction for a certain period of time or for a certain rotational distance, and this alternating rotation of the telescoping cutting assembly 104 may continue for the duration of the procedure or as may be desired by the user. When alternating rotation of the telescoping cutting assembly 104 in this manner, the cutting edges 130 of the telescoping cutting assembly 104 operate to “saw” the occlusive material from within a vessel, and the aspiration device 111 may provide negative pressure within the lumen 110 to suck or draw any dislodged plaque or thrombi particles proximally through the lumen 110 and out of the vessel. In embodiments, one or more of the cutting edges 130 of the telescoping cutting assembly 104 may be serrated. In embodiments, the intravascular cutting tool 100 may include one or more encoders (not illustrated) or sensors (not illustrated) for measuring the amount of rotational travel exhibited by the telescoping cutting assembly 104. For example, a controller (e.g., computer, chip, etc.) may be provided that is coupled to the one or more encoders or sensors and receives data therefrom indicative of a rotational position of the telescoping cutting assembly 104, and the controller may be communicatively coupled to the motor 206 and to thereby control rotation of the telescoping cutting assembly 104 based on the data.

FIG. 4 illustrates an example atherectomy or thrombectomy system 300 including the intravascular cutting tool 100 and an aspiration stylet 301, according to one or more embodiments shown and described herein. In the illustrated example, the distal end 101 of the intravascular cutting tool 100 is shown positioned proximate to an intervention site 302 within a vessel 304 having a blockage 306, wherein the blockage 306 may comprise built-up atheromatous plaque and/or clot or thrombus. As hereinafter described, the aspiration stylet 301 is utilizable to extract debris or particles 308 of plaque or thrombus that are broken off (or cut off) from the blockage 306 via actuation of the telescoping cutting assembly 104 as described herein.

The aspiration stylet 301 includes a body 310. The body 310 extends longitudinally along the axis A, from a distal end 312 to a proximal end (not shown). In embodiments, the aspiration stylet 301 may extend at least the entire length of the lumen 110, whereas, in other embodiments, the aspiration stylet 301 extends less than the entire length of the lumen 110. A plurality of aspiration inlets or openings 314 may be formed in the body 310 proximate to the distal end 312. Thus, the aspiration stylet 301 includes a perforated distal section. The body 310 is hollow and includes an internal lumen (not shown) extending there through that communicates with the plurality of openings 314. The aspiration device 111 may be disposed in communication with the lumen of the body 310 so as to apply a negative pressure within lumen of the body 310 and thereby create suction through the plurality of openings 314. In this manner, the aspiration stylet 301 it utilizable to suck and extract the particles 308 of the blockage 306 broken off via actuation of the telescoping cutting assembly 104.

In embodiments, a guidewire 316 may be utilized to facilitate positioning of the telescoping cutting assembly 104 and/or the aspiration stylet 301 relative to the intervention site 302. For example, the guidewire 316 may be provided through the lumen 110 of the intravascular cutting tool 100 to facilitate navigation of the telescoping cutting assembly 104. In embodiments utilizing the aspiration stylet 301, the guidewire 316 may extend through a lumen within the body 310 of the aspiration stylet 301. In embodiments, the guidewire 316 is utilized to adjust position of the telescoping cutting assembly 104. The guide wire 316 is utilizable to facilitate navigation to and from the intervention site 302 and, once at the invention site 302, the guidewire 316 may be removed. Upon removal of the guidewire 316, the aspiration stylet 301 can be distally advanced towards and through the blockage 306, such that the aspiration stylet 301 may thereafter be utilized to guide further advancement of the intravascular cutting tool 100 towards the blockage 306 and through the blockage 306 as the telescoping cutting assembly 104 cuts the blockage 306. As the telescoping cutting assembly 104 begins to cut the blockage 306, negative pressure is activated inside of the aspiration stylet 301 to collect debris (e.g., the particles 308 of the blockage 306). In embodiments, the guidewire 316 may remain in place as the telescoping cutting assembly 104 cuts the blockage 306 and the aspiration stylet 301 collects debris. In some embodiments, the guidewire 316 is not utilized.

The aspiration stylet 301 may be movable within the lumen 110. As illustrated as shown in FIG. 4, the aspiration stylet 301 may be inserted and advanced through the lumen 110 of the intravascular cutting tool 100 such that the distal end 312 of the body 310 extends distally beyond the opening 114 of the aspiration tip 112. In this manner, the aspiration stylet 301 is advanceable through the lumen 110 and may be extended through the blockage 306, such that the plurality of openings 314 formed in the body 310 of the aspiration stylet 301 are located on a distal side and a proximal side of the blockage 306, despite the telescoping cutting assembly 104 approaching the blockage 306 from the proximal side thereof. This allows the particles 308 broken off from the blockage 306 to be captured by the aspiration stylet 301 rather than flowing distally away from the intravascular cutting tool 100, for example, where blood flows distally away from the intravascular cutting tool 100. In embodiments, an exterior surface of the body 310 of the aspiration stylet 301 may include threads and an interior bore surface of the lumen 110 may include corresponding threads, such that the aspiration stylet 301 may be axially advanced or retracted along the axis A via rotating the body 310, which causes translation of the aspiration stylet 301 via the threaded engagement. In embodiments, the aspiration stylet 301 may be axially advanced towards and into the blockage 306 as the telescoping cutting assembly 104 remove more and more of the blockage 306 and open the vessel 304.

When inserting the intravascular cutting tool 100 into the subject and when navigating it to the intervention site 302, the aspiration stylet 301 may be at least partially retracted within the lumen 110 such that a distal end 312 of the body 310 is positioned proximal from the opening 114 of the aspiration tip 112. In embodiments, when navigating to the intervention site 302, the aspiration stylet 301 may be may be fully removed from the lumen 110, and then inserted into the lumen 110 once the telescoping cutting assembly 104 has been appropriately positioned proximate at the intervention site 302.

FIG. 4 also illustrates example operation of the intravascular cutting tool 100 during an atherectomy or thrombectomy procedure. After navigating the intravascular cutting tool 100 to the intervention site 302 with the telescoping cutting assembly 104 in the retracted configuration, the aspiration stylet 301 may be extended beyond the opening 114 of the aspiration tip 112 such that it is positioned to capture the particles 308 of the blockage 306. In embodiments, the aspiration stylet 301 is extended at least partially into the blockage 306; whereas in other embodiments, the aspiration stylet 301 is extended through the blockage 306. Thereafter, the aspiration device 111 is activated to create suction through the plurality of openings 314 and thereby capture the particles 308. Then, the pressure source 198 may be activated and apply the pressure P to the cavity 118 to move the first inner blade 124 and the second inner blade 126 into the cutting configuration, wherein the cutting edges 130 contact the blockage 306. This contact may result in breaking the particles 308 off from the blockage 306, which would then be aspirated out of the subject via the aspiration stylet 301. Then, the telescoping cutting assembly 104 may be rotated as indicated by the arrow 204. As mentioned, the telescoping cutting assembly 104 may rotate relative to the catheter 102 via the rotational bearing 108 or the telescoping cutting assembly 104 and the catheter 102 may rotate together. In embodiments, instead of rotating the telescoping cutting assembly 104, the pressure source 198 may cause the first inner blade 124 and the second inner blade 126 to longitudinally oscillate or reciprocate back and forth as indicated by the arrow 202. In embodiments, the telescoping cutting assembly 104 rotates as indicated by the arrow 204 and reciprocate back and forth as indicated by the arrow 202. Regardless of the motion(s) of the telescoping cutting assembly 104, the aspiration stylet 301 sucks away the particles 308 that are broken off of the blockage 306 via cutting action of the telescoping cutting assembly 104. After the telescoping cutting assembly 104 has removed the blockage 306 to a desired extent, negative pressure may be introduced to the cavity 118 to move the first inner blade 124 and the second inner blade 126 proximally into the retracted configuration (or an at least partially retracted configuration), such that the cutting edges 130 are less exposed, and then the intravascular cutting tool 100 is removed from the subject.

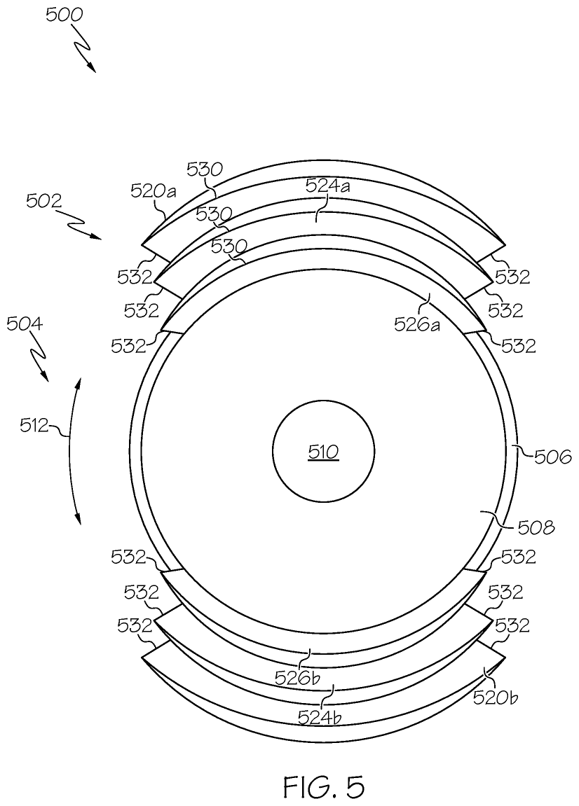

FIG. 5 illustrates an alternate intravascular cutting tool 500, according to one or more alternate embodiments shown and described herein. In particular, FIG. 5 illustrates a telescoping cutting assembly 502 disposed at a distal end 504 of the intravascular cutting tool 500, wherein the telescoping cutting assembly 502 comprises semi-circular blades. The intravascular cutting tool 500 also includes a catheter 506, and the telescoping cutting assembly 502 is provided on a distal end of the catheter 506. The catheter 506 may be similar to the catheter 102 described above. The intravascular cutting tool 500 also includes an aspiration tip 508 provided on the distal end of the catheter 506 and provided within an opening defined in the telescoping cutting assembly 502 as described above. Moreover, the intravascular cutting tool 500 includes a lumen 510 extending through the catheter 506 and the aspiration tip 508, which may operate as described above with reference to the aspiration tip 112 and the lumen 110 of FIGS. 1-4. Furthermore, as described above with reference to the telescoping cutting assembly 104, the telescoping cutting assembly 502 is rotatable (clockwise or counter-clockwise) as indicated by arrow 512. For example, in embodiments where the telescoping cutting assembly 502 is directly attached to the catheter 506, the telescoping cutting assembly 502 is rotatable via rotation of the catheter 506. However, where the telescoping cutting assembly 502 is coupled to the catheter 506 a rotational bearing, a high speed motor may be provided to rotate the telescoping cutting assembly 502 as indicated by the arrow 512.

In the illustrated embodiment, the telescoping cutting assembly 502 comprises a pair of outer blades 520a, 520b, a pair of first inner blades 524a, 524a nested within the pair of outer blades 520a, 520b, and a pair of second inner blades 526a, 526a nested within the pair of first inner blades 524a, 524a.

As described with reference to FIGS. 1-4, above, the pair of outer blades 520a, 520b may each be directly attached to a catheter body (e.g., the catheter 102) or may each be coupled to the catheter body via a rotational bearing (e.g., the rotational bearing 108). In embodiments, the pair of outer blades 520a, 520b are integrally attached to each other. For example, the pair of outer blades 520a, 520b may each be attached to a proximal base (e.g., a ring that is attached to the catheter) and the pair of outer blades 520a, 520b distally extend from the proximal base. In other embodiments, the pair of outer blades 520a, 520b are not attached to each other and are separate components that are each individually attached or coupled to the catheter.

As described above with reference to the first inner blade 124, the pair of first inner blades 524a, 524a are movable between the retracted configuration and the cutting configuration. In embodiments, the pair of first inner blades 524a, 524a are integrally attached to each other such that they are movable together, or the pair of first inner blades 524a, 524a may be separate blades that are independently movable relative to each other.

As described above with reference to the second inner blade 126, the pair of second inner blades 526a, 526a are movable between the retracted configuration and the cutting configuration. In embodiments, the pair of second inner blades 526a, 526a are integrally attached to each other such that they are movable together, or the pair of second inner blades 526a, 526a may be separate blades that are independently movable relative to each other.

The pair of first inner blades 524a, 524a and the pair of second inner blades 526a, 526a are movable, distally and proximally between the retracted configuration and the cutting configuration, as described above with reference to the first inner blade 124 and the second inner blade 126.

A cutting edge 530 is provided on each of the pair of outer blades 520a, 520b, the pair of first inner blades 524a, 524a, and the pair of second inner blades 526a, 526a. The cutting edges 530 are operable to cut a blockage upon distal movement of the pair of first inner blades 524a, 524a and the pair of second inner blades 526a, 526a.

Lateral cutting edges 532 may be formed on the sides of each of the pair of outer blades 520a, 520b, the pair of first inner blades 524a, 524a, and the pair of second inner blades 526a, 526a. The lateral cutting edges 532 are operable to cut a blockage upon (clockwise or counter-clockwise) rotation of telescoping cutting assembly 502 as indicated by the arrow 512.

From the above, it is to be appreciated that defined herein is an intravascular cutting tool that is able to cut and remove occlusive material from within a vessel, via axial movement and/or rotation movement, without cutting the endothelial layer.

Further aspects of the embodiments described herein are provided by the subject matter of the following clauses:
Clause 1. An intravascular cutting tool for an occlusion removal procedure, including: a catheter body; and a telescoping cutting assembly comprising an outer blade coupled to and extending distally from the catheter body and at least one inner blade positioned radially inward of the outer blade, the at least one inner blade being axially moveable between a retracted configuration and a cutting configuration, wherein the at least one inner blade extends more distally beyond the outer blade when in the cutting configuration than in the retracted configuration, and wherein the outer blade and the at least one inner blade cooperatively define a tapered cutting profile.
Clause 2. The intravascular cutting tool of clause 1, wherein the at least one inner blade and the outer blade are configured to rotate in the cutting configuration
Clause 3. The intravascular cutting tool of clause 2, wherein the at least one inner blade and the outer blade are rotatable relative to the catheter body.
Clause 4. The intravascular cutting tool of clause 3, further including a rotational bearing coupled to a distal end of the catheter body, wherein the outer blade is mounted to the rotational bearing.
Clause 5. The intravascular cutting tool of any preceding clause, wherein the catheter body defines an aspiration lumen extending there through, and an aspiration stylet is advanceable through the aspiration lumen and an opening defined through the at least one inner blade.
Clause 6. The intravascular cutting tool of any preceding clause, wherein the catheter body defines a pressure lumen and the telescoping cutting assembly defines an internal cavity in communication with the pressure lumen, and pressure communicated to the at least one inner blade through the pressure lumen and the internal cavity moves the at least one inner blade from the retracted configuration to the cutting configuration.
Clause 7. The intravascular cutting tool of any preceding clause, wherein the outer blade comprises a cutting edge that defines an outer cutting perimeter, and each of the at least one inner blades defines a cutting edge that is positioned radially inward of the outer cutting perimeter.
Clause 8. The intravascular cutting tool of any preceding clause, wherein the outer blade comprises one or more retention features that limit an axial motion of the at least one inner blade.
Clause 9. A method for atherectomy or thrombectomy procedure, including: advancing an intravascular cutting tool to an intervention site of a blood vessel, the intravascular cutting tool comprising: a catheter body, and a telescoping cutting assembly comprising an outer blade coupled to and extending distally from the catheter body and at least one inner blade positioned radially inward of the outer blade, the at least one inner blade being axially moveable between a retracted configuration and a cutting configuration, wherein the at least one inner blade extends distally beyond the outer blade when in the cutting configuration than in the retracted configuration, and wherein the outer blade and the at least one inner blade cooperatively define a tapered cutting profile; advancing the at least one inner blade to the cutting configuration; and reciprocating the at least one inner blade proximally and distally or rotating the telescoping cutting assembly to cut a lesion at the intervention site.
Clause 10. The method of clause 9, further comprising advancing an aspiration stylet through an aspiration lumen and through a center opening through the inner blade, the aspiration lumen extending through the catheter body.
Clause 11. The method of any preceding clause, further comprising rotating the telescoping cutting assembly while reciprocating the at least one inner blade.
Clause 12. The method of any preceding clause, wherein the telescoping cutting assembly is rotatable relative to the catheter body.
Clause 13. The method of any preceding clause, wherein the intravascular cutting tool further comprises a rotational bearing coupled to a distal end of the catheter body, wherein the outer blade is mounted to the rotational bearing.
Clause 14. The method of any preceding clause, further including moving the at least one inner blade from the retracted configuration to the cutting configuration via pressure communicated to the at least one inner blade through a pressure lumen defined in the catheter body.
Clause 15. The method of any preceding clause, wherein the outer blade comprises a cutting edge that defines an outer cutting perimeter, and each of the at least one inner blades defines a cutting edge that is positioned radially inward of the outer cutting perimeter.
Clause 16. The method of any preceding clause, wherein the outer blade comprises one or more retention features that limit an axial motion of the at least one inner blade.
Clause 17. A system for atherectomy or thrombectomy procedure, including: an intravascular cutting tool, including: a catheter body defining an aspiration lumen extending through the catheter body, and a telescoping cutting assembly comprising an outer blade coupled to and extending distally from the catheter body and at least one inner blade positioned radially inward of the outer blade, the at least one inner blade being axially moveable between a retracted configuration and a cutting configuration, wherein the at least one inner blade extends more distally beyond the outer blade when in the cutting configuration than in the retracted configuration, and wherein the outer blade and the at least one inner blade cooperatively define a tapered cutting profile; and an aspiration stylet advanceable through the aspiration lumen and an opening through the telescoping cutting assembly.
Clause 18. The system of clause 17, wherein the aspiration stylet defines a plurality of openings along a perimeter of the aspiration stylet.
Clause 19. The system of any preceding clause, further including a guidewire, wherein the aspiration stylet defines a stylet lumen extending there-through, and the guidewire is advanceable through the stylet lumen, the aspiration lumen, and the opening.
Clause 20. The system of clause 19, wherein the guidewire is configured to adjust positioning of the telescoping cutting assembly.

In should now be understood that embodiments of the present disclosure are directed to intravascular cutting tools operable to remove occlusive material via several different cutting motions. For example, the intravascular cutting tools may include a telescoping cutting assembly having an outer blade and at least one inner blade, wherein the at least one inner blades are operable to reciprocate towards and away the occlusive material to thereby repeatably stab or chop the occlusive material. Further, the telescoping cutting is rotatable, such that the outer blade and the at least one inner blade are operable to slice the occlusive material, and this rotation of the telescoping cutting may be performed simultaneously with cutting movement of the at least one inner blades. Accordingly, the intravascular cutting tools are operable to dislodge occlusive material via multiple cutting maneuvers that may be performed simultaneously. Moreover, aspiration may be provided during performance of such cutting maneuvers to thereby extracting pieces of occlusive material that have been dislodged or cut-away from within a vessel via the intravascular cutting tool 100. Even further, the location at which such aspiration is applied relative to the occlusive material may be adjusted so as to ensure capture of dislodged particles and thereby inhibit occlusive particles from flowing away from the intravascular cutting tool into the subject's bloodstream.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

It will be apparent to those skilled in the art that various modifications and variations can be made to the embodiments described herein without departing from the scope of the claimed subject matter. Thus, it is intended that the specification cover the modifications and variations of the various embodiments described herein provided such modification and variations come within the scope of the appended claims and their equivalents.

1. An intravascular cutting tool for an occlusion removal procedure, comprising:
a catheter body; and
a telescoping cutting assembly comprising an outer blade coupled to and extending distally from the catheter body and at least one inner blade positioned radially inward of the outer blade, the at least one inner blade being axially moveable between a retracted configuration and a cutting configuration, wherein the at least one inner blade extends more distally beyond the outer blade when in the cutting configuration than in the retracted configuration, and wherein the outer blade and the at least one inner blade cooperatively define a tapered cutting profile.

2. The intravascular cutting tool of claim 1, wherein the at least one inner blade and the outer blade are configured to rotate in the cutting configuration.

3. The intravascular cutting tool of claim 2, wherein the at least one inner blade and the outer blade are rotatable relative to the catheter body.

4. The intravascular cutting tool of claim 3, further comprising a rotational bearing coupled to a distal end of the catheter body, wherein the outer blade is mounted to the rotational bearing.

5. The intravascular cutting tool of claim 1, wherein the catheter body defines an aspiration lumen extending there through, and an aspiration stylet is advanceable through the aspiration lumen and an opening defined through the at least one inner blade.

6. The intravascular cutting tool of claim 1, wherein the catheter body defines a pressure lumen and the telescoping cutting assembly defines an internal cavity in communication with the pressure lumen, and pressure communicated to the at least one inner blade through the pressure lumen and the internal cavity moves the at least one inner blade from the retracted configuration to the cutting configuration.

7. The intravascular cutting tool of claim 1, wherein the outer blade comprises a cutting edge that defines an outer cutting perimeter, and each of the at least one inner blades defines a cutting edge that is positioned radially inward of the outer cutting perimeter.

8. The intravascular cutting tool of claim 1, wherein the outer blade comprises one or more retention features that limit an axial motion of the at least one inner blade.

9. A method for atherectomy or thrombectomy procedure, comprising:
advancing an intravascular cutting tool to an intervention site of a blood vessel, the intravascular cutting tool comprising:
a catheter body, and
a telescoping cutting assembly comprising an outer blade coupled to and extending distally from the catheter body and at least one inner blade positioned radially inward of the outer blade, the at least one inner blade being axially moveable between a retracted configuration and a cutting configuration, wherein the at least one inner blade extends distally beyond the outer blade when in the cutting configuration than in the retracted configuration, and wherein the outer blade and the at least one inner blade cooperatively define a tapered cutting profile;
advancing the at least one inner blade to the cutting configuration; and
reciprocating the at least one inner blade proximally and distally or rotating the telescoping cutting assembly to cut a lesion at the intervention site.

10. The method of claim 9, further comprising advancing an aspiration stylet through an aspiration lumen and through a center opening through the inner blade, the aspiration lumen extending through the catheter body.

11. The method of claim 9, further comprising rotating the telescoping cutting assembly while reciprocating the at least one inner blade.

12. The method of claim 9, wherein the telescoping cutting assembly is rotatable relative to the catheter body.

13. The method of claim 9, wherein the intravascular cutting tool further comprises a rotational bearing coupled to a distal end of the catheter body, wherein the outer blade is mounted to the rotational bearing.

14. The method of claim 9, further comprising moving the at least one inner blade from the retracted configuration to the cutting configuration via pressure communicated to the at least one inner blade through a pressure lumen defined in the catheter body.

15. The method of claim 9, wherein the outer blade comprises a cutting edge that defines an outer cutting perimeter, and each of the at least one inner blades defines a cutting edge that is positioned radially inward of the outer cutting perimeter.

16. The method of claim 9, wherein the outer blade comprises one or more retention features that limit an axial motion of the at least one inner blade.

17. A system for atherectomy or thrombectomy procedure, comprising:
an intravascular cutting tool, comprising:
a catheter body defining an aspiration lumen extending through the catheter body, and
a telescoping cutting assembly comprising an outer blade coupled to and extending distally from the catheter body and at least one inner blade positioned radially inward of the outer blade, the at least one inner blade being axially moveable between a retracted configuration and a cutting configuration, wherein the at least one inner blade extends more distally beyond the outer blade when in the cutting configuration than in the retracted configuration, and wherein the outer blade and the at least one inner blade cooperatively define a tapered cutting profile; and
an aspiration stylet advanceable through the aspiration lumen and an opening through the telescoping cutting assembly.

18. The system of claim 17, wherein the aspiration stylet defines a plurality of openings along a perimeter of the aspiration stylet.

19. The system of claim 17, further comprising a guidewire, wherein the aspiration stylet defines a stylet lumen extending there-through, and the guidewire is advanceable through the stylet lumen, the aspiration lumen, and the opening.

20. The system of claim 19, wherein the guidewire is configured to adjust positioning of the telescoping cutting assembly.
